(19) United States
(12) Plant Patent Application Publication
(10) Pub. No.: US 2008/0184441 P1
(43) Pub. Date: Jul. 31, 2008

(54) NUF-76

(76) Inventors: Russell T. Nagata, Belle Glade, FL (US); Philip Busey, Davie, FL (US)

Correspondence Address: Florida Foundation Seed Producers, Inc., P.O. Box 309, Greenwood, FL, 32443, US

(21) Appl. No.: 11/890,834

(22) Filed: Aug. 8, 2007

Related U.S. Application Data
(60) Provisional application No. 60/879,996, filed on Jan. 10, 2007.

(51) Int. Cl. A01H 5/00

(52) U.S. Cl. PLT/392

(57) ABSTRACT

Perennial St. Augustinegrass having plant hopper and southern chinch bug insect resistance, narrow leaf blades and slower leaf extension rates thus requiring less frequent mowing.

### BACKGROUND OF THE INVENTION

This invention relates to a new and distinct St. Augustinegrass that is the result of a complex hybridization program to develop a dark green, fine bladed St. Augustinegrass. Parents were selected for darker green foliage color, narrow leaf width, low maintenance and resistance to common insect pests and disease. This genotype was first labeled as FA 1997-108 and evaluated as NUF-76. This selection was propagated vegetatively to provide planting stock for studying performance and distinguishing NUF-76 from other St. Augustinegrass cultivars.

### CROSS REFERENCE TO RELATED APPLICATION

This application claims the benefit of U.S. provisional patent, application No. 60/879,996 filed Jan. 10, 2007.

### SUMMARY OF INVENTION

NUF-76 is a fine bladed, dark green St. Augustinegrass, Stenotaphrum secundatum that is resistant to the southern chinch bug, Blissus insularis and the plant hopper, Liburnia pseudoseminigra. NUF-76 has slow leaf extension growth rates that result in the need for reduced frequency of mowing, there by saving on fuel necessary for lawn maintenance and for wear and tear of the lawn mower.

### BRIEF DESCRIPTION OF THE ILLUSTRATIONS

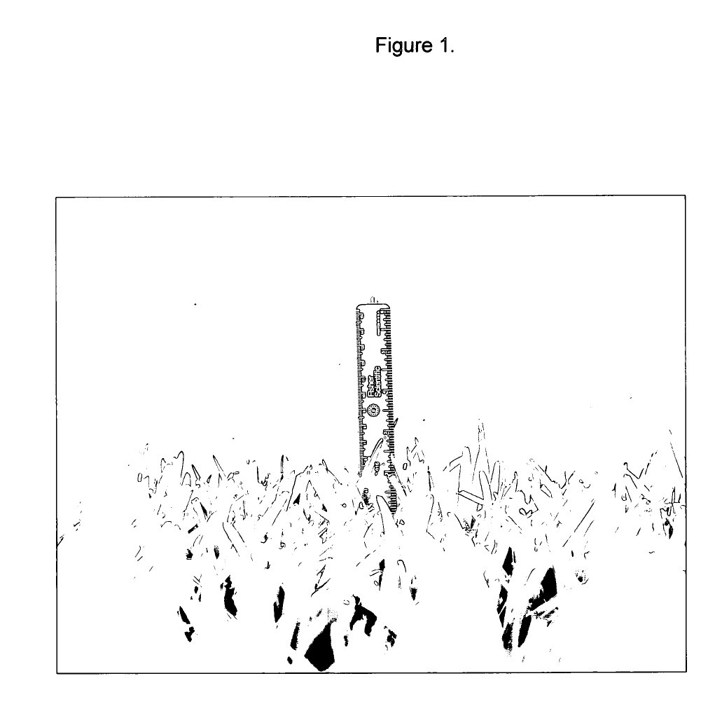

FIG. 1. NUF-76 St. Augustinegrass leaf blade height two weeks since mowing in experiments conducted in the month of July at University of Florida, IFAS, Everglades Research and Education Center.

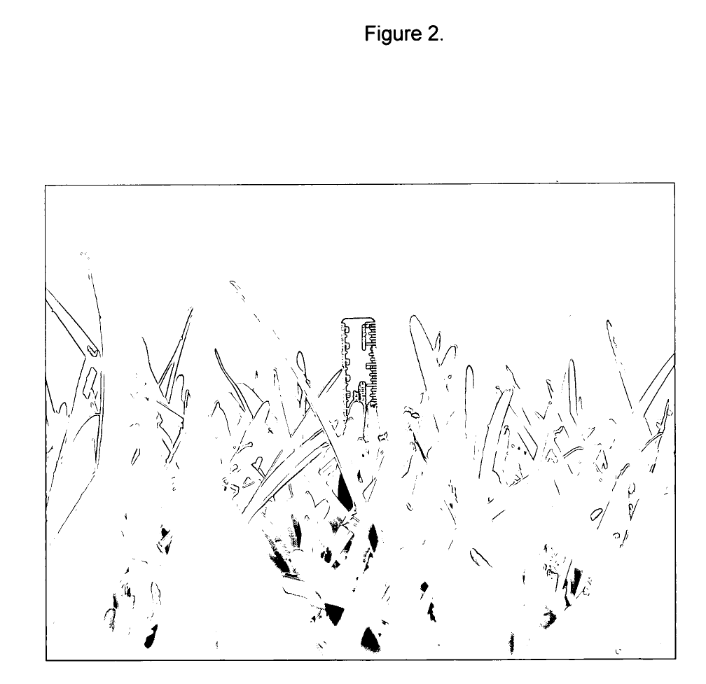

FIG. 2. Floratam St. Augustinegrass leaf blade height two weeks since mowing in experiments conducted in the month of July at University of Florida, IFAS, Everglades Research and Education Center.

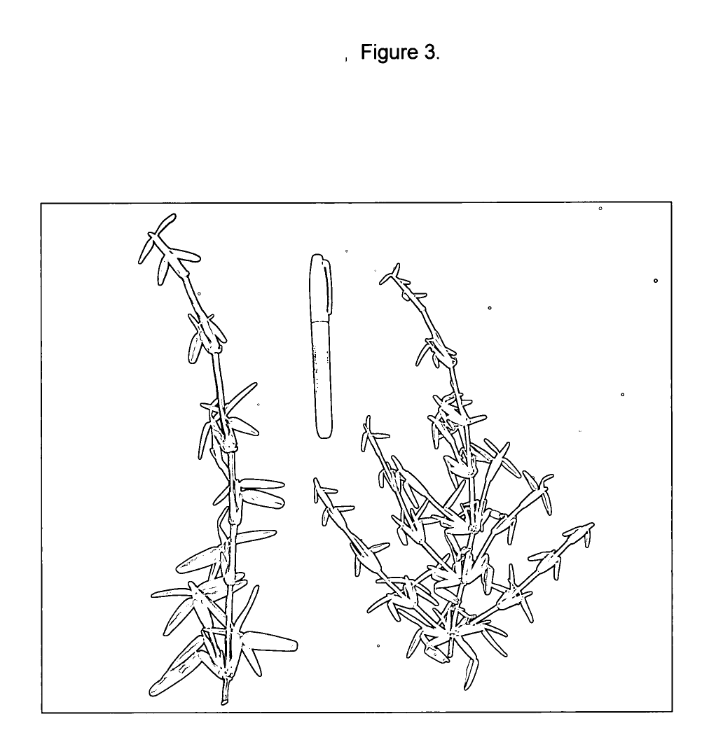

FIG. 3. Comparison of Floratam (left) and NUF-76 (right) runner growth and internode lengths.

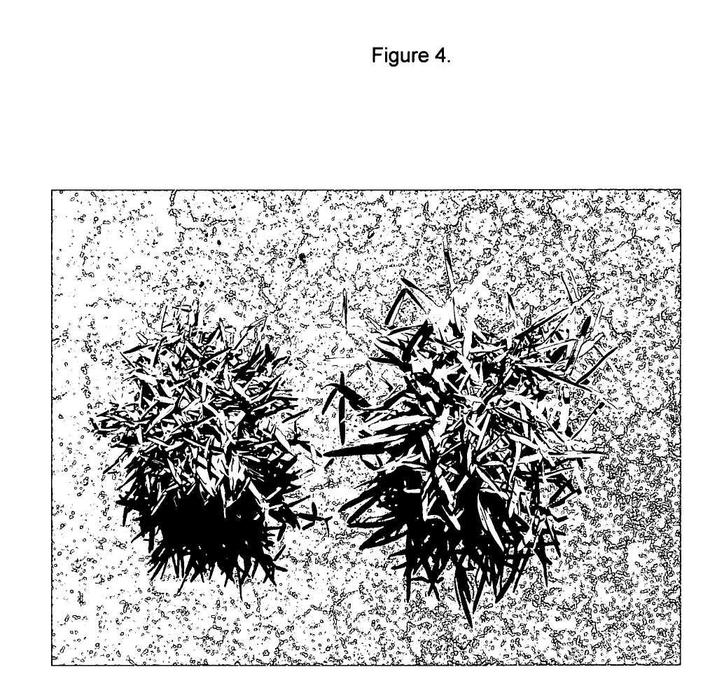

FIG. 4. Comparison of shoot and leaf density of Floratam (right) and NUF-76 (left).

### DETAILED DESCRIPTION OF THE VARIETY

### Insect and Disease Resistance

NUF-76 is resistant to two common insect pests, the southern chinch bug, Blissus insularis and the plant hopper (Tables 1 and 2). NUF 76 is the first St. Augustinegrass reported to have resistance to the plant hopper, Liburnia Pseudoseminigra. Plant hoppers feeding solely on NUF-76 survive 3.2 days on average compared with susceptible host plants in which they survived 11.2 or more days. Reproduction occurred on the susceptible host Classic and Palmetto, as evident by nymph production.

TABLE 1
Survival of Liburnia pseudoseminigra plant hopper on NUF-76 and
comparison St. Augustinegrasses².

| Variety/Selection | Days of Survival |
|---|---|
| Classic¹ | 18.2 a |
| Palmetto¹ | 13.0 ab |
| Bitterblue¹ | 11.2 b |
| Seville¹ | 8.2 bc |
| Floratam | 3.4 c |
| FX-10 | 3.4 c |
| NUF-216 | 3.2 c |
| FSHA-115 | 3.2 c |
| NUF-76 | 3.2 c |
| Floralawn | 2.6 c |

¹Plant hoppers survived past the 20 day duration of this experiment, at which point host plant fitness to support plant hopper survival diminishes.
²Means followed by the same letter are not significantly different (alpha = 0.05) using a protected LSD test (SAS 1996)

NUF-76 is resistant to all tested populations of southern chinch bugs from the state of Florida, including those that kill Floratam. For many southern chinch bug populations, mortality on NUF-76 was 100% within the 14 day test period. Chinch bug survival was significantly higher for Floratam and Floratine. Mortality on NUF-76 was similar to FX-10 and NUF-216, both selections having large leaves compared to NUF-76.

TABLE 2
Percent mortality of southern chinch bug, Blissus insularis feeding on
NUF-76 and comparison St. Augustinegrass held two weeks on
each cultivar. Chinch bugs collected from nine cities or their suburbs
in geographic regions of Florida¹.

| Variety | Mortality (%) | SD | Range |
|---|---|---|---|
| FX-10 | 94.9 a | 10.3 | 69-100 |
| NUF-216 | 94.6 a | 9.6 | 71-100 |
| NUF-76 | 91.3 a | 12.1 | 63-100 |
| Floratine | 60.6 b | 23.8 | 14-95 |
| Floratam | 47.2 b | 27.9 | 0-71 |

¹Means followed by the same letter are not significantly different (P = 0.05) using a protected LSD test (SAS 1996).

### Leaf Extension Rate

Leaf extension rate of NUF-76 was always shorter than all other St. Augustinegrass varieties, but was always statistically equal to Seville. Leaf extension rate of NUF-76 was equal to Seville and FX-10 and significantly less than NUF-216 and Floratam seven days after mowing. At 14 days after mowing, leaf growth was similar to Seville and FX-10. NUF-76 was half the height of Floratam and 60% the height of NUF-216. Three weeks after mowing, leaf growth of NUF-76 averaged 7.6 cm which was similar to Seville and 2.5 times shorter than Floratam. NUF-76 is shorter after 21 days of growth, than Floratam after 7 days. Using cultivars with slower leaf extension rates such as NUF-76 can reduce the frequency of mowing, thereby reducing the amount of fuel required to maintain a lawn and also reduce wear and tear of the mower. In a state such as Florida where more than 4 million acres of turfgrass is managed, elimination of a single mowing will result in significant fuel savings.

TABLE 3
Leaf blade growth of NUF-76 and comparison St. Augustinegrasses
one, two and three weeks after mowing¹.

Leaf Growth from Last
Mowing (cm)

| Variety | 7 days | 14 days | 21 days |
|---|---|---|---|
| NUF-76 | 3.8 c | 6.0 c | 7.6 d |
| Seville | 5.4 c | 6.3 c | 8.4 d |
| FX-10 | 4.6 c | 7.4 c | 10.3 c |
| NUF-216 | 7.2 b | 9.8 b | 13.6 b |
| Floratam | 9.6 a | 13.0 a | 19.0 a |

¹Means followed by the same letter are not significantly different (P = 0.05) using a protected LSD test (SAS 1996).

### Leaf Morphology

Unmowed NUF-76 has significantly shorter, narrowest, and smallest leaf area of comparison St. Augustinegrass cultivars. These reduced leaf characteristics result in NUF-76 appearance to be a fine leaf textured St. Augustinegrass with a dense and compact canopy. NUF-76 average leaf length measured 106.5 mm and a width of 6.7 mm. These measurements resulted in average leaf area of 62.6 square mm, 68% the area of its closest comparison variety.

TABLE 4
Measurements of leaf characteristics of NUF-76 and comparison St.
Augustinegrasses¹.

| Variety | Leaf Length (mm) | Leaf Width (mm) | Leaf Area (mm²) |
|---|---|---|---|
| NUF-76 | 106.5 d | 6.7 d | 62.6 d |
| Seville | 130.2 c | 8.0 c | 91.7 c |
| FX-10 | 153.2 b | 9.2 a | 122.3 b |
| NUF-216 | 170.2 b | 8.0 b | 116.7 b |
| Floratam | 204.4 a | 9.0 a | 154.7 a |

¹Means followed by the same letter are not significantly different (P = 0.05) using a protected LSD test (SAS 1996).

### Reproductive Structures

Inflorescences of NUF-76 are terminal and auxiliary, averaging 1.85 panicles per flowering culm. Mean spike length of NUF-76 is 97.75 mm, equal in shortness to 1997-6 and shorted than other cultivars. Floral region measurements averaged 57.35 mm for NUF-76 which was shorter than all cultivars except for FX-10. Spiklet or seed number per inflorescence was 25, equal to Floratam, Seville and Raleigh for the lowest number. Stigmas are white and anther sac color is yellow.

TABLE 5
Inflorescence characteristics NUF-76 and comparison St.
Augustinegrass¹.

| | | Floral | Number of | Spike |
|---|---|---|---|---|
| | Spike Length | Region | Spiklets | number per |
| Variety | (mm) | Length (mm) | (seeds) | shoot |
| FX-10 | 120.8 de | 59.0 d | 39.8 a | 2.3 bcd |
| NUF-216 | 161.8 a | 88.6 b | 36.0 a | 2.8 ab |
| NUF-232 | 132.6 cd | 84.4 b | 30.4 b | 2.7 abc |
| 1997-6 | 107.6 ef | 71.2 c | 29.5 b | 2.8 a |
| Floratam | 153.4 ab | 99.8 a | 28.6 bc | 2.1 d |
| Seville | 121.1 de | 68.6 c | 21.2 d | 2.2 cd |
| Raleigh | 142.4 bc | 71.3 c | 21.0 d | 2.2 cd |
| NUF-76 | 97.8 f | 57.4 d | 25.0 cd | 1.8 d |

¹Means followed by the same letter are not significantly different (P = 0.05) using a protected LSD test (SAS 1996).

TABLE 6
Anther and stigma color of NUF-76 and other comparison St.
Augustinegrass varieties¹.

| Variety | Anther Color | Stigma Color |
|---|---|---|
| NUF-76 | Yellow | White |
| Seville | Yellow | White hairs/purple shaft |
| Floratam | Orange Yellow | Purple |
| Raleigh | Yellow | White |
| Bitterblue | Orange Yellow | White hairs/purple shaft |
| Palmetto | Orange Yellow | White |

¹Color as observed at pollen dehiscence.

### Growth and Ground Coverage

Growth of NUF-76 lateral shoots was equal to other cultivars at 6 weeks from transplanting rooted cutting into the field. However, at 9 weeks lateral shoot lengths of NUF-76 was the shortest. Internode counts however did not differ among cultivars and reduce lateral length was attributed to shorter internodes. As a result, ground coverage during grow-in after sprigging or plugging a new field by NUF-76 was slowest for all cultivars tested. Starting with 3% coverage by the grass plug, mean coverage by NUF-76 was 27.8% after eight weeks. By 16 weeks of growth, all cultivars had 96% ground coverage or better indicating that coverage by NUF-76 will catch up to the other cultivars and coverage approach 100%.

TABLE 7
shoot length and node count of NUF-76 and comparison St.
Augustinegrass¹.

| | Shoot length | | Shoot length | Number of |
|---|---|---|---|---|
| | at 6 weeks | Number of | at 9 weeks | internodes 9 |
| Variety | (cm) | internodes 6 wks | (cm) | wks |
| NUF-76 | 10.1 a | 4.0 ab | 30.2 c | 8.4 a |
| Seville | 17.0 a | 4.6 a | 49.0 b | 9.5 a |
| FX-10 | 8.9 a | 2.5 b | 40.5 bc | 7.7 a |
| NUF-216 | 16.7 a | 3.8 ab | 61.2 a | 10.2 a |
| Floratam | 15.9 a | 4.6 a | 60.6 a | 10.2 a |

¹Means followed by the same letter are not significantly different (P = 0.05) using a protected LSD test (SAS 1996).

TABLE 8
Percentage coverage by NUF-76 and other comparison St.
Augustinegrasses. Planted Oct. 14, 2004 at University of Florida,
IFAS Everglades Research and Education Center on 30 cm spacing¹.

Percentage of Ground Cover by Turfgrass

| | 8 weeks | 12 weeks | 16 weeks |
|---|---|---|---|
| Variety | Dec. 13, 2004 | Jan. 19, 2005 | Feb. 22, 2005 |
| NUF-76 | 27.5 c | 56.2 c | 96.0 b |
| Floratam | 38.8 b | 83.8 b | 100.0 ab |
| NUF-216 | 48.8 a | 87.5 b | 98.5 a |
| Seville | 47.5 a | 94.2 a | 99.5 a |

¹Means followed by the same letter are not significantly different (P = 0.05) using a protected LSD test (SAS 1996).

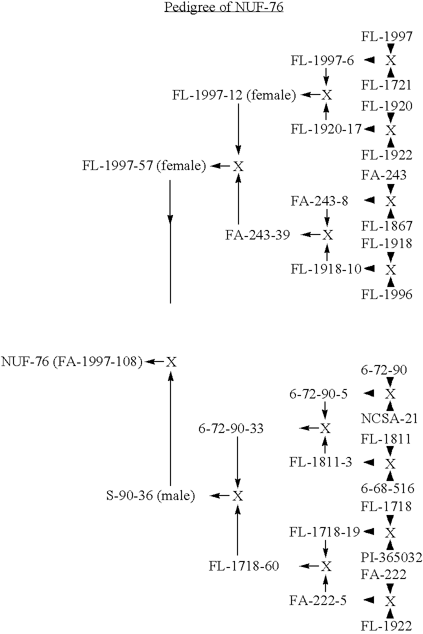

1. A new and distinct variety of St. Augustinegrass, substantially described and illustrated, characterized by its distinctive combination of insect resistance, vegetative characteristics and reduced mowing frequency.
